(19) United States
(12) Patent Application Publication
(10) Pub. No.: US 2026/0193982 A1
(43) Pub. Date: Jul. 9, 2026

(54) A DEVICE AND METHOD FOR MEASURING PRESSURE IN IMMISCIBLE FLUIDS IN A SUBTERRANEAN RESERVOIR

(71) Applicant: Hydrophilic AS, Tananger (NO)

(72) Inventors: Øivind Godager, SANDEFJORD (NO); Solveig RIISØEN, OLSVIK (NO); Trond Arne ROLFSVÅG, Tananger (NO); Harald Syse, RØYNEBERG (NO); Sondre Frantsen Tolo, Randaberg (NO)

(21) Appl. No.: 19/131,503

(22) PCT Filed: Nov. 15, 2023
(86) PCT No.: PCT/NO2023/060091

§ 371 Date: May 20, 2025

(30) Foreign Application Priority Data
Nov. 22, 2022 (NO) 20221251

(51) Int. Cl. E21B 49/08; E21B 47/06

(52) U.S. Cl. CPC E21B 49/0875; E21B 47/06; E21B 49/081

(57) ABSTRACT

A device (10a; 10b; 30) for measuring a first pressure (P_{O}) in a first fluid and a second pressure (P_{W}) in a second fluid, at a location in a hydrocarbon zone (Z_{O}) in a subterranean formation (9) comprises a water-filled system with a pressure-sensitive chamber (8a; 8b), a ceramic member (3), and a fluid conduit (7) fluidly connecting the pressure-sensitive chamber and the ceramic member, and a flow control device (5; 6; 12, 14; 41, 40) arranged in the fluid conduit at a location between the ceramic member and the pressure-sensitive chamber. A pressure sensor (4) is arranged between the ceramic member and the flow control device and configured and arranged for measuring the pressure (P_{M}) in the fluid conduit. The flow control device is operable to allow displacement of water through the ceramic member and towards at portion of the formation the outside the device. The invention comprises a dual pressure measurement system built around one pressure sensor. The invented apparatus uses one and same pressure sensor to measure two individual pressures in a subterranean reservoir at a given sensor location.

### TECHNICAL FIELD OF THE INVENTION

The invention concerns the exploration and production of hydrocarbons from subterranean reservoirs. More specifically, the invention is related to a device and a method for measuring pressure in immiscible fluids in a subterranean hydrocarbon reservoir.

### BACKGROUND OF THE INVENTION

Subterranean hydrocarbon reservoirs are normally found in or adjacent to aquifers. FIG. 1 is a schematic illustration of a reservoir R extending downwards in a formation from an upper boundary RT and comprising a hydrocarbon (e.g. oil) zone Z_{O} and a water zone Z_{W}. In the hydrocarbon zone, the oil pressure P_{O} is greater than the water pressure P_{W} (P_{O}>P_{W}) and vice versa in the water zone. A transition zone extends upwards from the oil-and-water interface (Oil-Water Contact: OWC). The OWC occurs where the oil pressure equals the sum of local formation pore entry pressure P_{E} and the formation water pressure P_{W} (P_{O}=P_{E}+P_{W}). The free water level FWL occurs at the level where the oil pressure equals the water pressure (P_{O}=P_{W}). Above the FWL, the reservoir may produce water, oil, or a mixture of oil and water. Determining the OWC and the FWL when exploring for hydrocarbons is important for accurately estimating the size of the discovery, and while producing hydrocarbons from a subterranean reservoir to avoid unwanted water production.

FIG. 1 illustrates a subterranean hydrocarbon reservoir in equilibrium, i.e. a static state in which the isobars are substantially horizontal. Therefore, FWL determined on the basis of measurements at one location (e.g. in well W_{A}), may be representative for the FWL at other locations within the same reservoir, such as at the well W_{B}.

However, once production of hydrocarbons commences, often through multiple wells in the same reservoir and supported by natural aquifers or by injection of water, the interface between oil and water becomes more complex, and it may be difficult to determine how the oil column is shrinking with time. FIG. 2 is a simplified and schematic illustration of a hydrocarbon reservoir a dynamic state, illustrating how the isobars are inclined. It should be understood that the isobars may have other inclinations than illustrated, as well as non-linear inclinations. The shape and properties of the underground formation may not be well known, and the water may invade the hydrocarbon reservoir in unpredictable ways. This makes it difficult to plan good locations for future wells, and may lead to drilling of wells that produce excessive amounts of water, add little value, and that might have to be re-drilled or permanently abandoned. Currently, a significant amount of data is collected to improve the prediction of the water movement. It is therefore a need for a system and method for more precisely determining the FWL at a desired location, as well as its variation over time, and as such determine the difference, ΔP, between the formation oil pressure, P_{O}, and the formation water pressure, P_{W}.

One disadvantage with the prior art is that formation water pressure and formation oil pressure are measured by respective individual sensors, which necessitates a subsequent calculation of respective pressure differences to estimate the distance to the free water level (water table).

The prior art includes U.S. Pat. No. 4,282,750, which discloses a system and a process for measuring the formation water pressure within an oil layer in a dipping reservoir. The system uses a modified “Repeat Formation Tester”, which contains a packer for forming a seal around a portion of a reservoir intersected by borehole and a backshoe for pushing the packer against the formation. A flowline is arranged for conducting fluid between the packer and a series of chambers, for containing fluid. Flows of fluid into or out of those chambers are individually controllable by valves in flowline. A pressure transducer is provided for measuring the pressure of the fluid in flowline, and an equalizing valve is arranged for opening the flowline into fluid communication with the fluid in the borehole. The flowline is connected to the packer via a probe within which there is a movable piston shaft and piston. The piston may be advanced until its end is sealed against the outer end of the probe by an O-ring.

The prior art also includes WO 2018/101838 A1, which discloses a probe and a probe arrangement for a pressure measurement of a water phase inside a hydrocarbon reservoir. The probe comprises a body comprising a pressure measuring chamber and at least one opening to the pressure measuring chamber, and a surface of the body is arranged with a hydrophilic characteristic. The probe arrangement comprises a displacement mechanism adapted to displace the probe from a first position, where the opening of the probe is located outside the reservoir, to a second position, where said at least one opening of the probe is located at a position inside the reservoir.

The prior art also includes WO 2020/236004 A1, which discloses a device for continuous water pressure measurement in a hydrocarbon reservoir, comprising a pressure sensor, a hydrophilic membrane positioned between a reservoir formation and the pressure sensor, the hydrophilic membrane having a surface area, and a biasing device pushing the hydrophilic membrane against the reservoir formation with a force equal to, or larger than, the pressure difference between a hydrocarbon phase in the reservoir and the water multiplied with the probe membrane contact area.

Background technology of some relevance is also described in WO 2022/182244 A1.

### SUMMARY OF THE INVENTION

The invention is set forth and characterized in the main claim, while the dependent claims describe other characteristics of the invention.

It is thus provided a device for measuring a first pressure in a first fluid and a second pressure in a second fluid, at a location in a hydrocarbon zone in a subterranean formation, characterized by:
a water-filled system, comprising a pressure-sensitive chamber, a ceramic member, and a fluid conduit fluidly connecting the pressure-sensitive chamber and the ceramic member, and a flow control device arranged in the fluid conduit at a location between the ceramic member and the pressure-sensitive chamber;
a pressure sensor arranged between the ceramic member and the flow control device and configured and arranged for measuring the pressure in the fluid conduit; and
wherein the flow control device is operable to allow displacement of water through the ceramic member and towards a portion of the formation outside the device.

In one embodiment, the flow control device comprises a valve or a pump. In another embodiment, the flow control device comprises a controllable heat source and a check valve, wherein the check valve is arranged between the pressure-sensitive chamber and the heat source. In another embodiment, the flow control device comprises an actuable member having a recess, whereby at least a portion of the water contained by the recess is injected into the fluid conduit when the actuable member is actuated. The actuable member may be a valve member and the recess may be defined by an O-ring.

In one embodiment, the pressure-sensitive chamber comprises a bladder or a bellows.

The ceramic member has hydrophilic properties and a low permeability compared to the permeability of the formation at said location in the formation.

The invented device may be used at a location in a hydrocarbon zone in a subterranean formation; wherein the first pressure is the formation oil pressure and the second pressure is the formation water pressure, and wherein the two pressures are measured by one and the same pressure sensor.

It is also provided a method of measuring a formation oil pressure and a formation water pressure at a location in a hydrocarbon zone in a subterranean formation, characterized by;
a) arranging and/or activating a water-filled system at said location in the formation, wherein the water-filled system comprises a pressure-sensitive chamber, a ceramic member, and a fluid conduit fluidly connecting the pressure-sensitive chamber and the ceramic member, and a flow control device arranged in the fluid conduit at a location between the ceramic member and the pressure-sensitive chamber;
b) arranging at least a portion of the ceramic member to abut against a portion of the formation, and exposing the pressure-sensitive chamber to the formation oil pressure,
c) activating a pressure sensor to continuously or periodically sense and record the pressure at a position in the fluid conduit between the flow control device and the ceramic member;
d) at a first point in time, activating the flow control device to displace at least a portion of water in the water-filled system through the ceramic member, while sensing and recording the pressure in the fluid conduit;
e) after a period of time following step d), de-activating the flow control device, and continue sensing and recording the pressure in the fluid conduit for a period of time.

Steps d) and e) may be repeated.

In one embodiment, step d) comprises opening of a valve, whereby said portion of water is displaced by the formation oil pressure exerted on the pressure-sensitive chamber. In another embodiment, step d) comprises the movement of an actuable member inside a space in the fluid conduit, said actuable member having a recess, whereby at least a portion of the water contained by the recess is injected into the fluid conduit.

### BRIEF DESCRIPTION OF THE DRAWINGS

These and other characteristics of the invention will become clear from the following description of various embodiments of the invention, given as non-restrictive examples, with reference to the attached schematic drawings, wherein:

FIG. 1 is a schematic illustration of a subterranean oil reservoir in a state of equilibrium;

FIG. 2 is a schematic illustration of an oil reservoir a dynamic state;

FIG. 3 and FIG. 4 are schematic illustrations of two variants of a first embodiment of the device according to the invention;

FIG. 5 is a diagram illustrating an operation of the device illustrated in FIGS. 3 and 4;

FIG. 6 is a schematic illustration of a second embodiment of the device according to the invention;

FIG. 7 is a diagram illustrating an operation of the device illustrated in FIG. 6;

FIG. 8 is a perspective view of a third embodiment of the device according to the invention;

FIG. 9 is sectional view along the central longitudinal axis of the device illustrated in FIG. 8, in a first state;

FIG. 10 is an enlarged view of area “A” in FIG. 9;

FIG. 11 corresponds to FIG. 10, but illustrates the device in a second state;

FIG. 12 is a perspective view of a valve assembly of the device illustrated in FIGS. 8-11; and

FIG. 13 is an enlarged view of area “B” in FIG. 10.

### DETAILED DESCRIPTION OF EMBODIMENTS OF THE INVENTION

The following description may use terms such as “horizontal”, “vertical”, “lateral”, “back and forth”, “up and down”, “upper”, “lower”, “inner”, “outer”, “forward”, “rear”, etc. These terms generally refer to the views and orientations as shown in the drawings and that are associated with a normal use of the invention. The terms are used for the reader's convenience only and shall not be limiting.

The invention comprises a dual pressure measurement system built around one pressure sensor. The invented apparatus uses one and same pressure sensor to measure two individual pressures in a subterranean reservoir (e.g. in a wellbore) at a given sensor location. One pressure measured is relating to hydrophilic water ingress at the given position, to estimate the distance to the free water level or water table, i.e. the formation water pressure, P_{W}. The other pressure measured is the true pressure in the formation at the location of the sensor, i.e. the formation oil pressure, or “Pressure in Oil”, P_{O}. An objective of the present invention is to be able to determine the difference between these two pressures, i.e. ΔP=P_{O}−P_{W} at the sensor location. Having determined ΔP at the sensor location and knowing the respective densities of water and oil, the distance to FWL (where ΔP=0) may be calculated by methods that are well known in the art.

One improvement over the prior art provided by this invention, is the elimination of systematic pressure measurement offset readings that will occur using two individual sensors. Thus, one sensor that read the two pressures will have much better precision to obtain the true pressure difference, than using two individual sensors and a subsequent calculation of respective pressure differences, to estimate the distance to FWL (i.e. the water table).

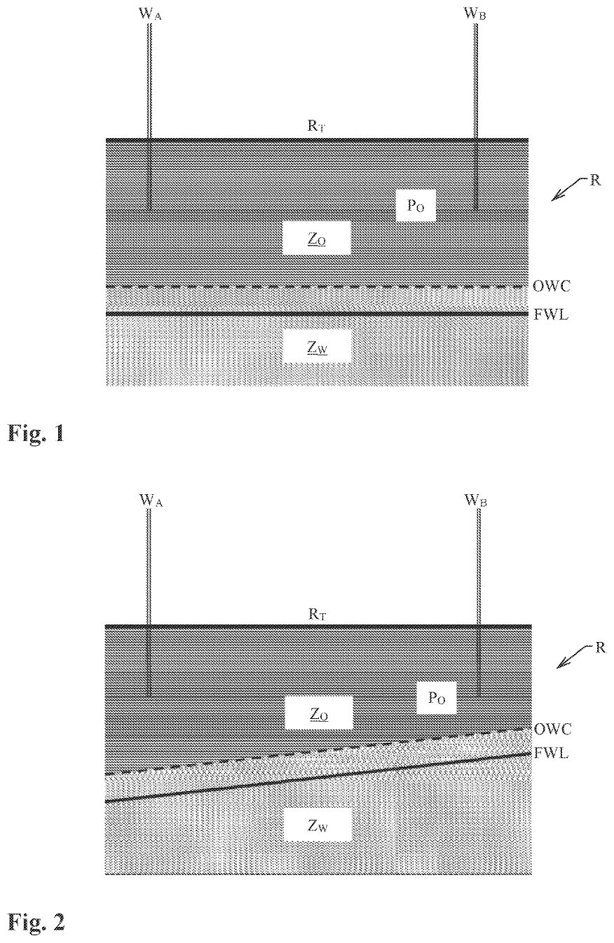

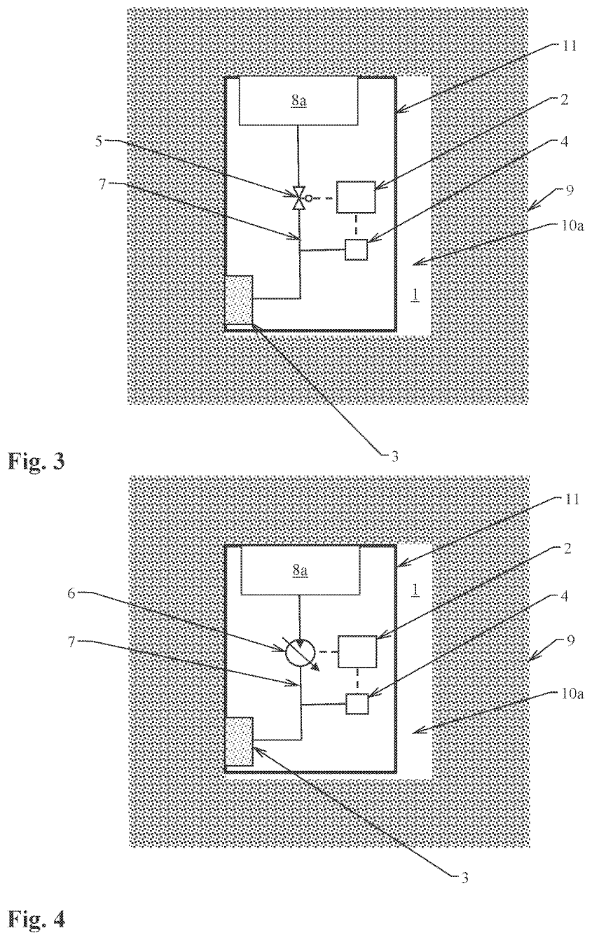

A first embodiment of the invention will now be described with reference to FIGS. 3-5. Referring to FIG. 3, a first embodiment of the invented device 10a comprises a housing 11 which is configured for installation in a cavity 1 in an oil reservoir (cf. Z_{O} in FIGS. 1 and 2) in a subterranean formation 9. The cavity may be a borehole or any other cavity in a formation in an oil reservoir, as described above with reference to FIGS. 1 and 2. The device 10a comprises a chamber 8a containing water (or a liquid having similar properties) The chamber 8a is a pressure-sensitive chamber, in that it responds to the ambient pressure around the device. As such, the pressure-sensitive chamber may comprise a bladder, a bellows, or other pressure-transferring device. Therefore, when the device is installed in the formation, the water in the pressure-sensitive chamber will be at the same pressure as that of the surrounding formation (i.e. P_{O}). A fluid conduit 7 connects the pressure-sensitive chamber 8a with a probe comprising a ceramic member 3 (or another similar material having a suitable pore size and distribution). The pressure-sensitive chamber 8a, the fluid conduit 7, and the ceramic member 3 may collectively be referred to as a water-filled system.

A valve 5 is arranged in the fluid conduit 7 between the ceramic member 3 and the pressure-sensitive chamber 8a, and is operable to control fluid flow between the ceramic member and the pressure-sensitive chamber. The valve 5 may be any valve known in the art and suited for the intended purpose, for example a solenoid valve. A pressure sensor 4 is connected to the fluid conduit 7 at a location between the ceramic member 3 and the valve 5, and is operable to measure the pressure in the fluid conduit 7 between the ceramic member 3 and the valve 5. Reference number 2 denotes a control, power, and communications module, which is connected to the valve and to the pressure sensor. It should be understood that parts and equipment necessary operate the device at a downhole location are not shown, as such items and techniques are well known in the art.

The probe tip comprising the ceramic member 3 is arranged such that the ceramic member is bearing against the formation, as illustrated in FIG. 3. When the valve 5 is opened, water flows into and through the ceramic member 3 by virtue of the pressure difference between the pressure-sensitive chamber 8a and the ceramic member. As water present in the reservoir above the OWC (Oil-Water Contact) generally has very low mobility, a comparably long time will normally pass before a true state of equilibrium is achieved between the reservoir and the ceramic member (and hence the water-filled system). Therefore, introducing water in this manner into the ceramic member 3 in a quantity sufficient to bring the water saturation in the ceramic member above equilibrium with the reservoir, contributes to assist and expedite a process towards equilibrium (and thus achieve a shorter response time).

FIG. 4 illustrates a variant of the embodiment illustrated in FIG. 3, in which the valve 5 has been replaced by a pump 6 (e.g. a piezoelectric pump).

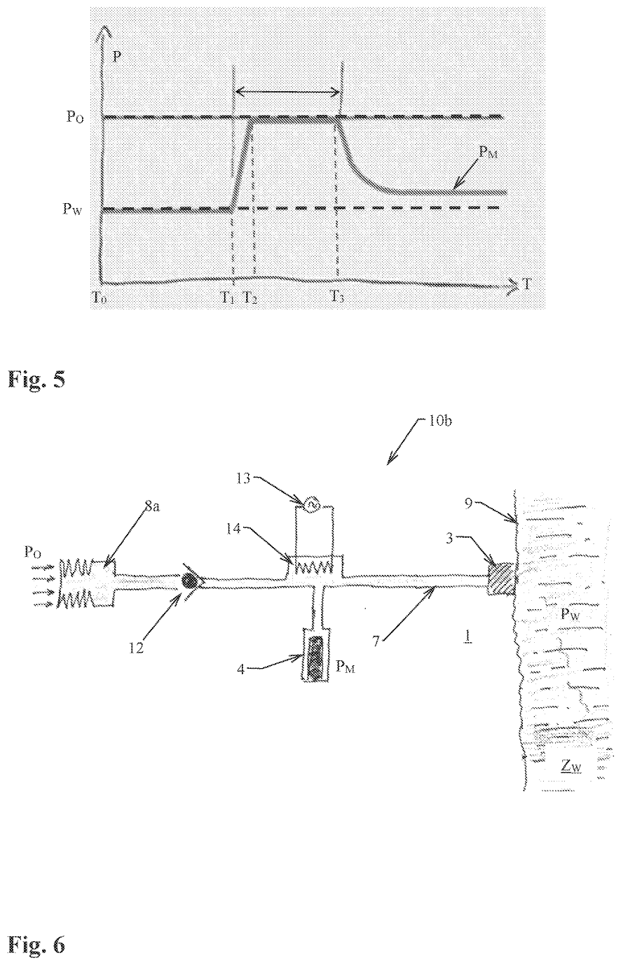

FIG. 5 illustrates the measurement procedure made possible with the invented device. The pressure P_{M} as measured by the pressure sensor 4 over a period of time T is drawn as a solid line. The dotted line at P_{O} is the formation oil pressure and the dotted line at P_{W} is the formation water pressure, both at the location where the device is installed. The diagram in FIG. 5 shows that, during period of time after installing the pressure sensing system in the formation (at T₀), the pressure sensor will measure pressure P_{W} (P_{M}=P_{W}). At T₁, the valve 5 is opened (or—in the alternative illustrated in FIG. 4: the pump 6 is activated), which will cause water to flow to the ceramic member 3 due to P_{O} acting on the water in the pressure-sensitive chamber 8a. The measured pressure P_{M} will thus be seen as rising due to the water influx into the ceramic member 3 and the relative low permeability in the ceramic member 3. At T₂, pressure build-up settles off, because of the pressure balance between P_{O} and P_{W}. Here P_{M} in the front end of the water-filled system (between the valve (or pump) and the ceramic member) equals the formation pressure P_{O}. As pressure settlement is observed with the valve open (or pump activated), the pressure sensor 4 measures the true formation pressure P_{O}. After establishing P_{O}, the valve is closed (or pump deactivated) at T₃. The water volume in the conduit 7 will the reduce due to flow through the ceramic member. The measured pressure P_{M} continues to drop as water is feed through the ceramic member 3, and will over time approach the hydrophilic water pressure P_{W}.

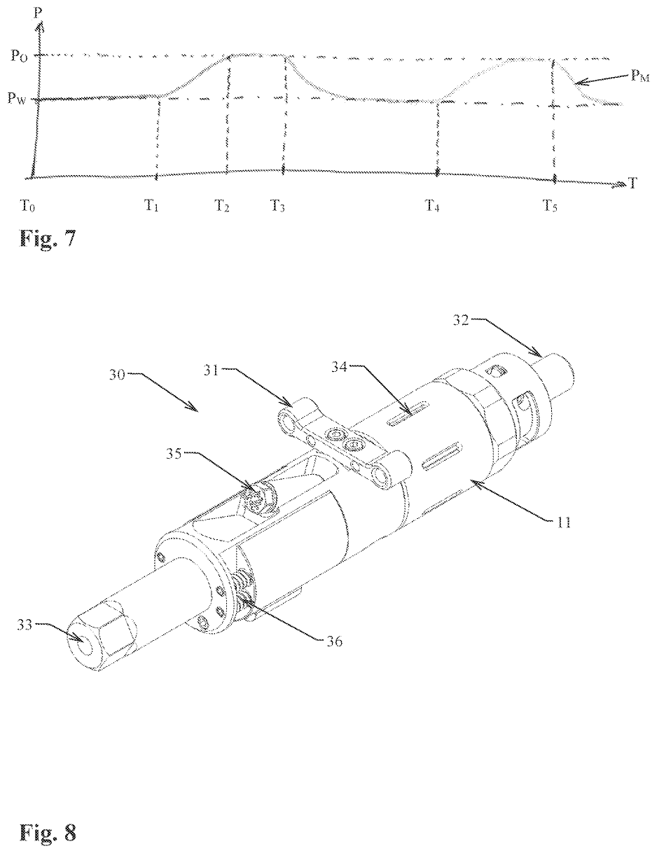

A second embodiment of the invention will now be described with reference to FIGS. 6 and 7. Although not illustrated, it shall be understood that this embodiment of the device also comprises a housing configured for being arranged in a cavity in a subterranean formation, and a control, power and communications module as described above with reference to the first embodiment. And unless otherwise specified, parts, features and functions described above with reference to the first embodiment shall be applicable also to this second embodiment. It should furthermore be understood that parts and equipment necessary operate the device at a downhole location are not shown, as such items and techniques are well known in the art.

Like the device 10a of the first embodiment, the device 10b of the second embodiment also comprises a water-filled system comprising a pressure-sensitive chamber 8a and a ceramic member 3 interconnected by a fluid conduit 7. The water inside the pressure-sensitive chamber 8a is thus at the same pressure as that of the surrounding formation, P_{O}.

In this second embodiment of the invention, the device 10b comprises a controllable heat source 14 which is configured and arranged to heat water inside the fluid conduit 7. The heat source may be an electrically powered heating element or member, connected to an electric power source 13, but the invention shall not be limited to such heat source. A check valve 12 is arranged in the fluid conduit 7, between the heat source 14 and the water-filled pressure-sensitive chamber 8a. A check valve is a preferred valve type, as the device may be permanently installed in the formation and a check valve does not require any external power source to operate. A pressure sensor 4 is connected to the fluid conduit 7 at a location between the ceramic member 3 and the check valve 12, and is operable to measure the pressure in the fluid conduit 7 between the ceramic member 3 and the check valve 12. Pressure measured by the pressure sensor 4 is designated P_{M}. In the illustrated embodiment, the heat source is arranged in the vicinity of the pressure sensor.

Operating the heat source 14 enables the device 10b to be switched between two pressure measurement modes: A first mode, measuring the water pressure, P_{W}, and a second mode, measuring the “Pressure in Oil”, P_{O}. The advantage of this is that the switch phase will allow the higher formation pressure P_{O} to feed water into the water-filled system and the pressure build-up will in turn systematically purge and clean the ceramic member 3 at the probe tip to enable continuous and firm hydrophilic water contact to the water table (FWL). The latter will also help to avoid contamination of the ceramic member 3. Further, using a heat source to switch between the two operating modes will reduce power requirement of the overall system in applications where power and space is scarce. The heat source makes the invented device simple and robust and adds to reliability with time and reduces operational risk related to the prior art method of switching the system to measure two independent pressures.

In the first mode, the pressure sensor 4 measures the hydrophilic pressure P_{W}. The pressure sensor 4 will measure the capillary pressure in the system through the ceramic member 3 towards the free water level (or water table) FWL. As the formation pressure P_{O} surrounding the device 10b at the given location in the wellbore is higher than P_{W}, the check valve 12 will normally be closed in the direction towards the pressure sensor 4. After the device 10b has been installed in the formation, the pressure in the water-filled system will align itself with P_{W} with time through the ceramic member 3. After a period the pressure sensor 4 will read the hydrophilic pressure P_{W}. The time this takes is depending on the permeability of the ceramic member 3 and the effective water permeability in the formation.

In the second mode, the pressure sensor 4 measures the pressure at the sensor location P_{O}. FIG. 7 illustrates the pressure P_{M} (solid line) as measured by the pressure sensor 4, over a period of time T. The dotted line at P_{O} is the oil pressure and the dotted line at P_{W} is the formation water pressure, both at the location where the device is installed. The diagram in FIG. 7 shows that, during period of time after installing the pressure sensing system in the formation (at T₀), the pressure sensor will measure pressure P_{W} (P_{M}=P_{W}). At T₁, the heat source is activated and will heat water inside the fluid conduit. The heated water will cause pressure in the front end of the water-filled system (i.e. between the check valve 12 and the ceramic member 3) to increase due to the expansion of volume in water as the temperature rises. The measured pressure P_{M} will thus be seen as rising due to the heat supplied to the water and pressure builds up in the system because of the relative low permeability in the ceramic member 3. At T₂, pressure build-up settles off, because of the pressure balance between P_{O} and P_{W}. Here P_{M} in the front end of the water-filled system equals the formation pressure P_{O}. Excess heat now will just feed the pressure-sensitive chamber 8a as it is in balance with the formation 9. As pressure settlement is observed with the heat source turned on, the pressure sensor 4 measures the true formation pressure P_{O}. After establishing P_{O} with the heat source on, the heat source is turned off at T₃. The water volume in the conduit 7 will in turn reduce due to the heat loss. This will then enable the pressure-sensitive chamber 8a to feed water to the conduit 7 as the heat reduces, and at some point the check valve 12 will close. The measured pressure P_{M} continues to drop as water is feed through the ceramic member 3, and at a certain point it will again settle or balance with the hydrophilic water pressure P_{W}. FIG. 7 shows how this procedure may be repeated at T₄ and T₅, illustrating how pressure measurements can be performed at desired intervals.

This second embodiment is particularly suited for a device permanently installed in a reservoir, as solenoid valves and piezoelectric pumps are energy-demanding.

The invented device (both first and second embodiments) is particularly useful for monitoring a producing hydrocarbon reservoir over an extended time period, for example several years. Power and data may be transferred between the device and an uphole location in any manner known in the art, for example wirelessly.

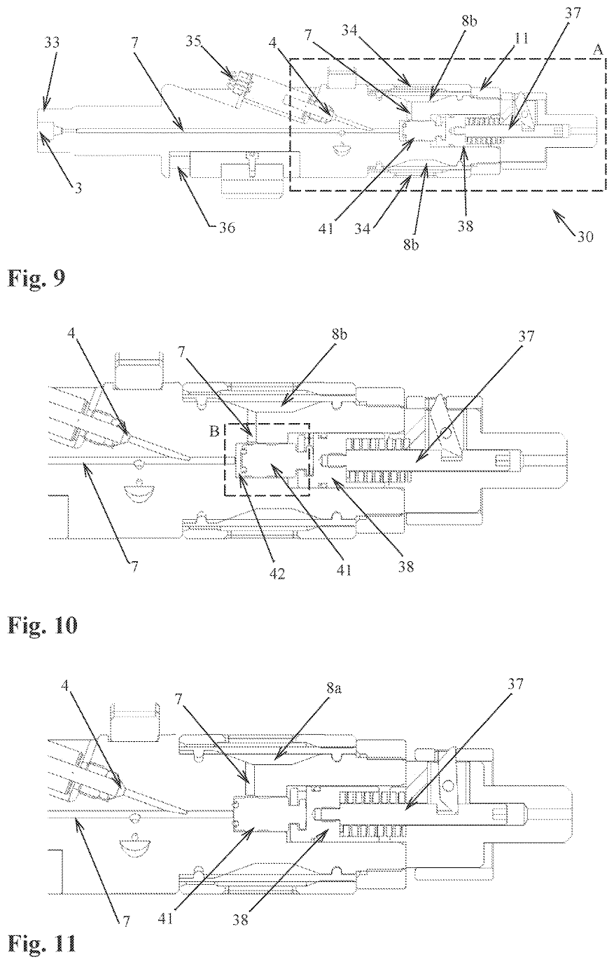

A third embodiment of the invention will now be described with reference to FIGS. 8-13. Referring initially to FIG. 8, the device 30 according to the invention comprises a housing 11 which in the illustrated embodiment has a cylindrical shape and is configured for connection to a wellbore tool or assembly (not shown), for example via a guiding-and-connection structure 31. The device 30 may be carried and operated by a wellbore tool known in the art, for example the arrangement described by the above-mentioned WO 2018/101838 A1. Reference number 35 denotes a connector interface for a pressure sensor 4 (see FIG. 9), while reference number 32 denotes a device-to-tool interface via which the device 30 may be operated.

Referring to FIGS. 9 and 10, the device 30 comprises a probe tip 33 which is configured for being placed against a portion of a subterranean formation, for example in a pre-drilled borehole adjacent to a wellbore. The probe tip holds a ceramic member 3, similar to the ceramic member described above in the first and second embodiments of the invention. A pressure-sensitive chamber 8b is arranged inside the housing 11 and is exposed to the formation (and P_{O}) through openings 34 in the housing wall, in the same manner as described above with reference to the first and second embodiments. A fluid conduit 7 connects the pressure-sensitive chamber 8b with a ceramic member 3 arranged at the probe tip 33, and a valve member 41 is arranged to selectively and controllably block or open the fluid conduit 7, by moving inside a space 42 in the fluid conduit 7. The valve member 41 is part of a valve assembly 38 which design and operation is well known in the art, and need therefore does not need to be described in detail. Reference number 37 denotes a valve actuator mechanism.

A pressure sensor 4 is fluidly connected to the conduit 7 at a location between the valve member 41 and the ceramic member 3. The pressure-sensitive chamber 8b, the fluid conduit 7 (including the space 42), and the ceramic member 3 comprise a water-filled system, similar to in the first and second embodiments of the invention.

FIG. 10 illustrates the valve member 41 in an open position, allowing fluid displacement (i.e. water flow) between the pressure-sensitive chamber 8b and the ceramic member 3. FIG. 11 illustrates the valve member 41 in a closed position, preventing fluid flow between the pressure-sensitive chamber 8b and the ceramic member 3.

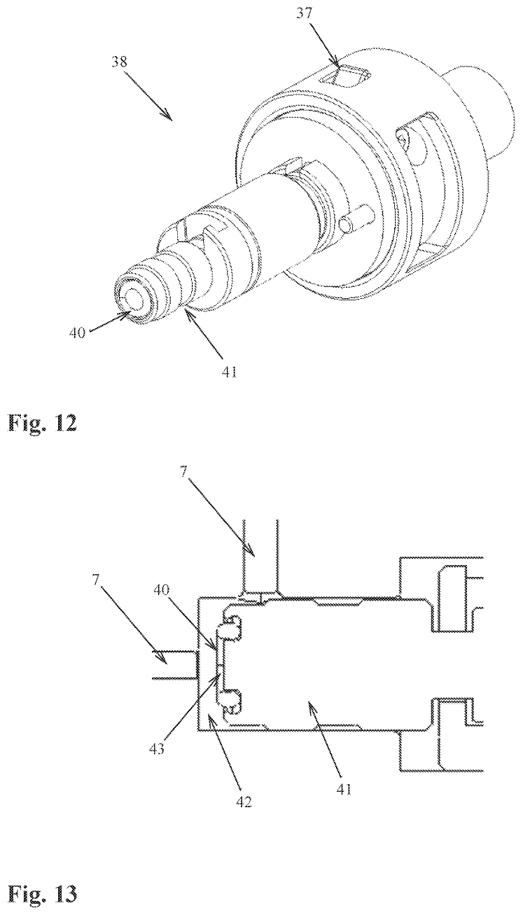

Referring to FIG. 12 and FIG. 13, an O-ring 40 is arranged at the tip of the valve member 41 and thus defines a recess 43 on the valve member. It should be understood that a recess 43 may be defined by other devices and structures on the valve member. When the valve member 41 is advanced inside the space 42 (towards the left in FIG. 13) to close the valve, the recess 43 traps a small volume of water, and some of this water is injected into the fluid conduit and out through the ceramic member 3 as the valve closes.

The device 30 in this third embodiment is configured for being introduced into a borehole and have the probe tip abut against the formation. The device therefore comprises a shock absorber mechanism whereby the impact between the probe tip and the formation may be reduced. Reference number 36 denoted spring members associated with this mechanism. The device may comprise a designated drilling device (not shown) integrated with the probe tip or as a separate device.

When installing the device 30 in a borehole (not shown), prior to the pressure measurement procedure, the valve member 41 is in a closed position (FIG. 11). When the valve closes, the O-ring is squeezed against the wall inside the space 42, whereby the volume of water trapped in the recess 43 is injected into the fluid conduit 7, and a pressure spike can be measured by the pressure sensor 4. Based on the dimensions and the permeability of the ceramic member 3, the flow rate and the volume of water over the ceramic member can be calculated by using Darcy's law of permeability. The surface of the ceramic member will, after the valve has closed, have a small amount of mobile water on its surface. If the probe tip (and thus that part of the ceramic member 3 facing the formation) is surrounded by oil, the pressure sensor 4 will read the formation oil pressure P_{O}. When the probe tip 33 touches for example a porous rock surface (the reservoir), a second pressure spike might be measured by the pressure sensor, and a second pulse of water flows through the ceramic member. When the probe tip 33 is lifted off the formation surface and the valve member 41 is opened, another pressure drop is measured.

With oil present, the pressure sensor 4 will be able to read both the oil and water pressure inside the reservoir, as explained above with reference to the first and second embodiments. If the valve member 41 is opened and closed again after the probe tip has been retracted away from the formation surface, the surrounding oil pressure can be re-confirmed (as mobile water on the surface of the ceramic member will transmit the surrounding oil pressure to the sensor).

A commonality between the three embodiments of the device 10a, 10b, 30 as described above, is the use of a single pressure sensor to measure two individual pressures (P_{W}, P_{O}) in a subterranean reservoir at a given sensor location by displacing water from a water-filled system through a ceramic member 3 which is placed against the formation in the reservoir. As the volume of the water-filled system is known, as well as the dimensions and permeability of the ceramic member, the water flow rate and volume across the ceramic member may be calculated, using Darcy's law of permeability. The surface of the ceramic member will be provided with a small amount of mobile water on its surface. If the ceramic member is surrounded by oil, the pressure sensor will read the oil pressure.

In the device 10a according to the first embodiment, water is displaced through the ceramic member by opening a valve or operating a pump. In the device 10b according to the second embodiment, the water displacement is caused by thermal expansion of water. In these two embodiments, the formation oil pressure P_{O}—by its action on the pressure-sensitive chamber—is utilized to drive water through the ceramic member. However, in the device 30 according to the third embodiment, water is not displaced through the ceramic member by the formation oil pressure P_{O}, but the volume of water trapped by the recess 43 and forced towards the ceramic member when the valve member is moved to its closed position. As the volume of the recess is known, so is the displaced volume of water, and pressure is measured when the system is in a state of equilibrium.

The pressure difference ΔP at the location of the device in the formation may be calculated from the sensed water pressure P_{W} and the sensed oil pressure P_{O}, as described above. It should be understood that the oil pressure P_{O} at the location of the device may be sensed by a sensor other that the sensor 4 in the device.

1. A device for measuring a first pressure in a first fluid and a second pressure in a second fluid, at a location in a hydrocarbon zone in a subterranean formation, the device comprising:
a water-filled system, comprising a pressure-sensitive chamber, a ceramic member, and a fluid conduit fluidly connecting the pressure-sensitive chamber and the ceramic member, and a flow control device arranged in the fluid conduit at a location between the ceramic member and the pressure-sensitive chamber;
a pressure sensor arranged between the ceramic member and the flow control device and configured and arranged for measuring the pressure in the fluid conduit; and
wherein the flow control device is operable to allow displacement of water through the ceramic member and towards a portion of the formation outside the device.

2. The device of claim 1, wherein the flow control device comprises a valve or a pump.

3. The device of claim 1, wherein the flow control device comprises a controllable heat source and a check valve wherein the check valve is arranged between the pressure-sensitive chamber and the heat source.

4. The device of claim 1, wherein the flow control device comprises an actuable member having a recess, whereby at least a portion of the water contained by the recess is injected into the fluid conduit when the actuable member is actuated.

5. The device of claim 4, wherein the actuable member is a valve member and the recess is defined by an O-ring.

6. The device of claim 1, wherein the pressure-sensitive chamber comprises a bladder or a bellows.

7. The device of claim 1, wherein the ceramic member has hydrophilic properties and a low permeability compared to the permeability of the formation at said location in the formation.

8. Use of the device of claim 1, at a location in a hydrocarbon zone in a subterranean formation, wherein the first pressure is the formation oil pressure and the second pressure is the formation water pressure, and wherein the two pressures are measured by one and the same pressure sensor.

9. A method of measuring a formation oil pressure and a formation water pressure at a location in a hydrocarbon zone in a subterranean formation (9), the method comprising:
a) arranging and/or activating a water-filled system at said location in the formation, wherein the water-filled system comprises a pressure-sensitive chamber, a ceramic member, and a fluid conduit fluidly connecting the pressure-sensitive chamber and the ceramic member, and a flow control device arranged in the fluid conduit at a location between the ceramic member and the pressure-sensitive chamber;
b) arranging at least a portion of the ceramic member to abut against a portion of the formation, and exposing the pressure-sensitive chamber to the formation oil pressure;
c) activating a pressure sensor to continuously or periodically sense and record the pressure at a position in the fluid conduit between the flow control device and the ceramic member;
d) at a first point in time, activating the flow control device to displace at least a portion of water in the water-filled system through the ceramic member, while sensing and recording the pressure in the fluid conduit; and
e) after a period of time following step d), de-activating the flow control device, and continue sensing and recording the pressure in the fluid conduit for a period of time.

10. The method of claim 9, wherein steps d) and e) are repeated.

11. The method of claim 9, wherein step d) comprises opening of a valve, whereby said portion of water is displaced by the formation oil pressure exerted on the pressure-sensitive chamber.

12. The method of claim 9, wherein step d) comprises movement of an actuable member inside a space in the fluid conduit, said actuable member having a recess, whereby at least a portion of the water contained by the recess is injected into the fluid conduit.
